(19) United States
(12) Patent Application Publication
(10) Pub. No.: US 2003/0148927 A1
(43) Pub. Date: Aug. 7, 2003

(54) Stable solutions of peptide copper complexes and cosmetic and pharmaceutical formulations produced therefrom

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/264,427

(22) Filed: Oct. 4, 2002

Related U.S. Application Data
(60) Provisional application No. 60/327,371, filed on Oct. 5, 2001.

(51) Int. Cl. A61K 38/16

(52) U.S. Cl. 514/6

(57) ABSTRACT

Compositions are formed by adding at least one amino acid to an aqueous solution of at least one peptide copper complex, and disclosed methods impart to solutions of peptide copper complexes, chemical stability and resistance to precipitation of the complexes through the addition of at least one of certain amino acids thereto. Also disclosed are compositions comprising at least one peptide copper complex and at least one of certain amino acids, as well as compositions further comprising one or more preservatives to provide thereto resistance to microbial attack and toxicity to microbes. Medical devices, pharmaceuticals and cosmetics comprising such compositions are also disclosed.

### CROSS-REFERENCE TO RELATED APPLICATION

This application claims the benefit of U.S Provisional Patent Application No. 60/327,371 filed Oct. 5, 2001, which application is incorporated herein by reference in its entirety.

### BACKGROUND OF THE INVENTION

1. Field of the Invention

The present invention relates to aqueous solutions of peptide copper complexes, and to pharmaceutical and cosmetic preparations, as well as medical devices, comprising such solutions.

2. Description of the Related Art

Copper is known to have many beneficial biological applications, including, as a few examples, stimulating the accumulation of collagen and elastin, increasing the rate of wound healing, and increasing the amount of collagen in skin (see, e.g., Maquart, F. X., Pickart, L., Laurent, M., Gillery, P., Monboisse, J. C., Borel, J. P., “Stimulation of Collagen Synthesis in Fibroblast Cultures by the Tripeptide-Copper Complex Glycyl-L-Histidyl-L-Lysine-Copper(2+),” FEBS Lett. 238(2): 343-346,1988; Wegrowski, Y., Maquart, F. X. and Borel, J. P., “Stimulation of Sulfated Glycosaminoglycan Synthesis by the Tripeptide-Copper Complex Glycyl-L-Histidyl-L-Lysine-Copper(2+),” Life Sciences 51: 1049-1056, 1992; Maguart, F. X., Bellon, G., Chaqour, B., Wegrowski, J., Patt L. M., Trachy, R. E., Monboisse, J. C., Chastang, F., Birembaut, P., Gillery, P. and Borel, J. P., “In Vivo Stimulation of Connective Tissue Accumulation by the Tripeptide-Copper Complex Glycyl-L-Histidyl-L-Lysine-Copper(2+) in Rat Experimental Wounds,” J. Clin. Invest. 92: 2368-2376, 1993).

Copper salts alone are ineffective, or even inhibitory, for such applications. The copper must be delivered in a biologically acceptable form. As an example, when copper is complexed with a biologically acceptable carrier molecule, such as a peptide, it may then be effectively delivered to cells.

More specifically, peptide copper complexes may be effective in this regard. Peptide copper complexes that are useful for wound healing and skin health are disclosed in U.S. Pat. Nos. 4,760,051; 4,665,054; 4,877,770; 5,135,913 and 5,348,943. Peptide copper complexes, beneficial for stimulating hair growth and preventing hair loss, are disclosed in U.S. Pat. Nos. 5,177,061; 5,214,032; 5,120,831; 5,550,183 and 5,538,945. Another beneficial application of peptide copper complexes is the prevention and healing of gastric ulcers, as disclosed in U.S. Pat. Nos. 5,145,838; 4,767,753 and 5,023,237. Yet another utility of such complexes is the healing of bone, as disclosed in U.S. Pat. No. 5,059,588.

While a number of peptide copper complexes have been identified and described as having biologically beneficial utility, there remains a need in the art for such solutions that can be more effectively, economically and easily used, alone or as a component of pharmaceuticals, medical devices, or cosmetic products. More specifically in this regard, needed in the art are solutions of peptide copper complexes that are chemically stable, that maintain the complexes in solution, even at higher concentrations and lower temperatures, and that resist microbial growth. Also needed in the art are pharmaceuticals, medical devices, or cosmetic products that comprise such solutions of peptide copper complexes. The present invention fulfills these needs and provides further related advantages.

### BRIEF SUMMARY OF THE INVENTION

In one embodiment, the present invention provides compositions formed from adding at least one amino acid to an aqueous solution of at least one peptide copper complex, with the proviso that the amino acid is not histidine. It has been surprisingly found that such compositions, by virtue of the addition of the at least one amino acid, are characterized by the peptide copper complex having an enhanced tendency to remain solubilized despite being present at relatively high concentrations in a solution maintained at lower temperatures. It has also been surprisingly found that such compositions are further characterized by exhibiting chemical stability, also by virtue of addition of the at least one amino acid.

In another embodiment, there is disclosed adding a preservative to the above composition to impart thereto greater resistance to microbial attack, as well as toxicity to microbes. Surprisingly, it has been found that compositions of the present invention are resistant to microbial attack and are toxic to microbes without the addition of a preservative, where the peptide copper complex is present in solution at a sufficiently high concentration.

Other embodiments are directed to compositions that comprise an aqueous solution of at least one peptide copper complex and at least one amino acid, with the proviso that the amino acid is not histidine. Yet additional embodiments are directed to such compositions that further comprise a ternary complex, the latter formed from the reaction of the peptide copper complex and the amino acid.

The present invention is also directed to a method for enhancing the chemical stability of compositions that contain an aqueous solution of a peptide copper complex, by adding at least one amino acid to the solution. Also disclosed is a method to enhance the ability of peptide copper complexes to remain in solution, despite being present at a relatively high concentration in an aqueous solution maintained at a relatively low temperature or frozen and then thawed, by adding at least one amino acid to the aqueous solution of the at least one peptide copper complex.

Finally, in another embodiment, the present invention is also directed to a medical device that comprises a disclosed composition.

These and other aspects of the present invention will be evident upon reference to the following detailed description of the invention.

### DETAILED DESCRIPTION OF THE INVENTION

As noted above, in one embodiment, disclosed is a composition formed by adding at least one amino acid to an aqueous solution of at least one peptide copper complex, with the proviso that the amino acid is not histidine. In certain specific embodiments, the at least one amino acid is glycine, lysine, argenine or a mixture thereof. Also, in certain other specific embodiments, the at least one peptide copper complex is glycyl-histidyl-lysine:copper(II) (“GHK-Cu”), valyl-histidyl-lysine:copper(II) (“VHK-Cu”) or alanyl-histidyl-lysine:copper(II) (“AHK-Cu”). Such peptides may be in either the L or D form. In a related, more specific embodiment, they are all in the L form.

As used herein, the term “peptide copper complex”refers to a coordination compound comprising a peptide molecule and a copper ion non-covalently complexed therewith. The peptide molecule serves as the complexing agent by donating electrons to the copper ion to yield the non-covalent complex. The peptide molecule is a chain of two or more amino acid units covalently bonded together via amide linkages (for example, —CONH—), the formation of such linkages being accompanied by the elimination of water. The amino acid units are from amino acids that are naturally occurring or otherwise. Also, at least one amide linkage nitrogen atom may have covalently bonded thereto either a hydrogen atom or another moiety.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units of the peptide copper complexes comprised in the compositions of the present invention may be provided by amino acids other than alpha-amino acids. For example, the amino acids may be beta- or gamma-amino acids, such as those shown below.

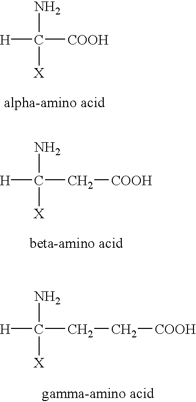

where X is the amino acid side-chain moiety.

Naturally occurring amino acids, that is, amino acids from which the amino acid units of naturally occurring proteins are derived, and their respective naturally occurring, amino acid side chain moieties, are shown below in Table 1. These naturally occurring amino acids are all in the L configuration, referring to the optical orientation of the alpha carbon or other carbon atom bearing the amino acid side chain. A peptide molecule may also comprise amino acids that are in the D optical configuration.

TABLE 1
NATURALLY OCCURRING AMINO ACID SIDE-CHAIN
MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH₂CH(CH₃)₂ | Isoleucine |
| —(CH₂)₄NH₃⁺ | Lysine |
| —(CH₂)₃NHC(NH₂)NH₂⁺ | Arginine |
| 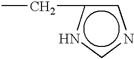 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 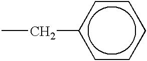 | Phenylalanine |
| 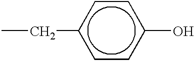 | Tyrosine |
| 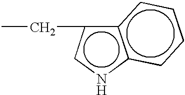 | Tryptophan |
| —CH₂SH | Cysteine |
| —CH₂CH₂SCH₃ | Methionine |
| —CH₂OH | Serine |
| —CH(OH)CH₃ | Threonine |

One example of a copper peptide complex is alanyl-histidyl-lysine:copper(II). Copper(II), as is well understood by the skilled artisan, designates a copper ion having a valence of 2 (e.g., Cu⁺²). Additional examples of peptide copper complexes, at least some of which are encompassed in embodiments of the present invention, include, but are not limited to, those described in U.S. Pat. Nos. 4,665,054; 4,760,051; 4,767,753; 4,877,770; 5,023,237; 5,059,588; 5,120,831; 5,135,913; 5,145,838; 5,177,061; 5,214,032; 5,348,943; 5,538,945 and 5,550,183.

Further, the expression “peptide copper complex,” as used herein, encompasses peptide copper complex derivatives. The expression “peptide copper complex derivative,” as used herein, refers to a peptide copper complex where the peptide molecule thereof has: 1) at least one amino acid side chain moiety that is a modification and/or variation of a naturally occurring, amino acid side-chain moiety; and/or 2) at least one of the hydrogens, bonded to an amide linkage nitrogen atom, substituted with a different moiety; and/or 3) the carboxyl group of the carboxyl terminal residue esterified or otherwise modified; and/or 4) at least one hydrogen, bonded to the nitrogen atom of the amino-terminal residue, substituted with a different moiety.

The amino acid side-chain moieties of the peptide copper complex derivatives may include alkyl, aryl, arylalkyl, alkoxy, or aryloxy moieties. As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl,” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like). As some examples, the amino acid side-chain moieties of alanine, valine, leucine, isoleucine and phenylalanine may generally be classified as alkyl, aryl or arylalkyl moieties.

“Alkoxy” and “aryloxy,” as used herein, refer, respectively, to alky and aryl moieties, as defined above, but each further comprising an oxygen atom used to link the moiety to the amino acid.

Additionally, the peptide copper complex derivative may, for example, be N-alkylated at one or more peptide bonds; and/or its carboxyl terminus may be esterified, for example, with a methyl, ethyl, or benzyl group, or may be reduced to a hydroxy or aldehyde. Additionally, the peptide copper complex derivative may, for example, be N-alkylated, N-acylated or N-sulfonylated at the amino terminus with, for example, methyl, benzyl, acetyl, benzoyl, methanesulfonyl, or fluorenyloxycarbonyl moieties.

Examples of the peptide copper complex derivatives, encompassed in embodiments of the present invention, include, but are not limited to, those disclosed and described in the above-cited U.S. Patents that are directed to peptide copper complexes, as well as those disclosed and described in the published PCT application having the international publication number WO 94/03482.

As one specific example, a disclosed composition may comprise a peptide copper complex derivative that is a derivative of GHK-Cu having the general formula:

[glycyl-histidyl-lysine-R]: copper(II)

where R is an alkyl moiety containing from 1 to 18 carbon atoms, an aryl moiety containing from 6 to 12 carbon atoms, an arylalkyl moiety, an alkoxy moiety containing from 1 to 12 carbon atoms, or an aryloxy moiety containing from 6 to 12 carbon atoms. This derivative of GHK-Cu is further described in the above-cited U.S. Patents that are directed to peptide copper complexes.

The above compositions may be prepared from aqueous solutions of peptide copper complexes. Such solutions are prepared by methods that are well known to those skilled in the art. For example, an amount of dried peptide copper complex suitable for a desired concentration is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH.

The molar ratio of the at least one peptide copper complex to the at least one amino acid, for embodiments directed to the above-disclosed compositions, ranges from 1:1 to 3:1. In one specific embodiment, the molar ratio is about 1:1. In various embodiments, the concentration of the at least one peptide copper complex, by weight of solution, is in the range of about 1% to about 25%, about 5% to about 15%, and about 7% to about 10%, respectively. Also, in some embodiments, the pH of the solution of the at least one peptide copper complex is adjusted by adding an acid. Typically, the acid used is HCl. However, other acids may be used, such as other inorganic acids, or organic acids. In certain embodiments, the adjusted pH is in the range of about 4.5 to about 7.2.

In another embodiment, at least one preservative is added to the above-disclosed, representative composition to impart resistance thereto to microbial attack and toxicity to microbes. Examples of such added preservatives include, but are not limited to, diazolidinyl urea, benzyl alcohol phenoxyethanol, imidazolidinyl urea, iodopropynyl butylcarbamate, sodium hydroxymethyglycinate, methylparaben, propylparaben, isopropylparaben, n-butylparaben, isobutylparaben, or a mixture thereof. Other preservatives, including mixtures thereof, may be used, as is appreciated by those skilled in the art. In yet another embodiment, the composition further comprises propylene glycol. The latter is used to dissolve the above parabens when they are at high concentrations.

As previously noted, it has unexpectedly been found that when the at least one peptide copper complex is present in solution at sufficiently high concentrations, the compositions of the present invention are not only resistant to microbial attack, but toxic to microbes, without the addition of a preservative. This is the case when the concentration of the at least one peptide copper complex is at least 7.5% by weight of solution. Also, as previously noted, it has been surprisingly found that compositions, formed by adding at least one amino acid to an aqueous solution of at least one peptide copper complex (as disclosed above), are chemically stable, by virtue of adding the at least one amino acid. As used herein, “chemical stability” refers to the ability of a composition to maintain at least 90% of its pharmaceutical or desired chemical activity for a desired period of time.

It has also been surprisingly found, as noted previously, that adding at least one amino acid to an aqueous solution of at least one peptide copper complex (as disclosed above), enhances the ability of the at least one peptide copper complex to remain solubilized. As a more specific example, it has been found that, at peptide copper complex concentrations of up to at least 10% by weight of solution, the addition of glycine or lysine to the solution can prevent precipitation of the at least one peptide copper complex. This has been found when the solutions are maintained at ambient temperatures (15° C. to 30° C.), as well as at refrigeration temperatures (2° C. to 8° C.) for extended periods of time (up to three weeks). This has also been found when the solutions are frozen (at −15° C. to −20° C.) and then thawed.

In another embodiment, the present invention is directed to a composition comprising an aqueous solution of at least one peptide copper complex and at least one amino acid, with the proviso that the amino acid is not histidine. In more specific, related embodiments, the amino acid is glycine, lysine, arginine or a mixture thereof. In another related embodiment, the composition further comprises a ternary complex formed from the at least one peptide copper complex and the at least one amino acid. An example of such a ternary complex, formed when glycine is added to an aqueous solution of GHK-Cu, is:

glycyl-histidyl-lysine:copper(II):glycine

As can be appreciated by one skilled in the art, an active drug substance may be combined with a disclosed composition of the present invention to provide a pharmaceutical preparation. The latter would typically also comprise an inert and physiologically-acceptable carrier or diluent, such as water, physiological saline, bacteriostatic saline, a petrolatum based cream, a pharmaceutically acceptable gel, a short chain alcohol, or a short chain glycol

Further, a disclosed composition may be combined with a suitable agent to provide a cosmetic preparation, as would be appreciated by one skilled in the art. Such agents may include, for example, a sunscreen agent, a skin-conditioning agent, a tanning agent, a skin protectant, an emollient, a humectant, or a mixture thereof. Also, such cosmetic preparations may further include an emulsifying agent, a surfactant, a thickening agent, an excipient, or a mixture thereof, as well as other additives, including: a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof.

Accordingly, in another embodiment, a disclosed composition is in the form of a liquid, lotion, cream, gel, emulsion, or microemulsion.

In another aspect, the present invention is directed to a method for imparting chemical stability to a composition comprising an aqueous solution of at least one peptide copper complex by adding to the solution at least one amino acid. In specific related embodiments, the at least one amino acid added is glycine, lysine, argenine or a mixture thereof, and the molar ratio of the at least one peptide copper complex to the at least one amino acid is at least about 1:1.

Also disclosed is a method for preventing the precipitation of peptide copper complexes from aqueous solutions of the same by adding at least one amino acid to the solution. Specific embodiments are directed to such a method where the at least one amino acid added is glycine, lysine, argenine or a mixture thereof, and the molar ratio of the at least one peptide copper complex to the at least one amino acid is at least about 1:1. The latter disclosed methods are directed to preventing precipitation of the peptide copper complexes at ambient (15° C. to 30° C.) or refrigeration temperatures (2° C. to 8° C.), or when the solution is frozen (at −15° C. to −20° C.) and then thawed.

Finally, in another aspect, the present invention is directed to medical devices that comprise a disclosed composition. One example of such a device is a sterile gauze pad, impregnated with a disclosed composition in the form of a gel for application to a wound.

The following examples, which illustrate the preparation, characterization, and utility of certain embodiments of the present invention, are provided for the purpose of illustration, not limitation. For all examples, concentrations are expressed as a percentage by weight of the solution.

### EXAMPLES

### Example 1

Solutions of glycyl-L-histidyl-L-lysine:copper(II) complex were prepared at a concentration of 10% with and without the addition of an amino acid. In addition, the pH of one sample was decreased to 4.5.

| Ingredients | Concentration |
|---|---|
| Solution 1A: | |
| Purified Water | q.s. 100% |
| Glycyl-L-Histidyl-L-Lysine: Copper(II) | 10% |
| complex | |
| PH | 6.7 |
| Solution 1B: | |
| Purified Water | q.s. 100% |
| Glycyl-L-Histidyl-L-Lysine: Copper(II) | 10% |
| complex | |
| Glycine | 1.8% |
| PH | 6.5 |
| Solution 1C: | |
| Purified Water | q.s. 100% |
| Glycyl-L-Histidyl-L-Lysine: Copper(II) | 10% |
| complex | |
| L-Lysine | 5.2% |
| PH | 6.5 |
| Solution 1D: | |
| Purified Water | q.s. 100% |
| Glycyl-L-Histidyl-L-Lysine: Copper(II) | 10% |
| complex | |
| PH | 4.5 |

After formulation, the solutions were filtered through 0.2 micron filters and stored under three conditions of temperature: ambient (room temperature), refrigerated, and frozen. After three days, the samples were examined (after complete thawing in the case of the frozen samples).

Solubility Results after 3 Days

| | Room | | |
|---|---|---|---|
| | Temperature | Refrigerated | Freeze-Thaw |
| Solution 1A | Clear deep | Deep blue | Deep blue solution. |
| | blue solution, | solution. | Lots of precipitate |
| | no precipitate | Precipitate on | on bottom. |
| | | bottom. | |
| Solution 1B | Clear deep | Clear deep | Deep blue solution. |
| | blue solution, | blue solution, | Some precipitate |
| | no precipitate | no precipitate | on bottom. |
| Solution 1C | Clear deep | Clear deep | Clear deep blue |
| | blue solution, | blue solution, | solution, no |
| | no precipitate | no precipitate | precipitate |
| Solution 1D | Clear deep | Clear deep | Deep blue solution. |
| | blue solution, | blue solution, | Some precipitate |
| | no precipitate | no precipitate | on bottom. |

The addition of a L-lysine to the solution of peptide copper complex prevented the formation of a precipitate upon storage at refrigerated conditions or upon freezing followed by thawing. The addition of glycine also prevented formation of a precipitate upon storage at refrigerated conditions, but allowed some precipitation upon freezing followed by thawing. Lowering the pH to 4.5 also aided in the prevention of precipitate formation.

### Example 2

The solutions of Example 1 were stored for an additional 3 weeks at room temperature and at refrigerated conditions. The following results were observed.

Solubility Results after 3 Weeks

| | Room Temperature | Refrigerated |
|---|---|---|
| Solution 1A | Deep blue solution. | Deep blue solution. Lots of |
| | Precipitate on bottom, | precipitate on bottom. |
| Solution 1B | Clear deep blue solution, | Clear deep blue solution, |
| | no precipitate | no precipitate |
| Solution 1C | Clear deep blue solution, | Clear deep blue solution, |
| | no precipitate | no precipitate |
| Solution 1D | Clear deep blue solution, | Clear deep blue solution, |
| | no precipitate | no precipitate |

The addition of a L-lysine or glycine to the solution of peptide copper complex prevented the formation of a precipitate upon storage at refrigerated conditions. Lowering the pH to 4.5, instead of adding an amino acid, also aided in the prevention of precipitate formation.

### Example 3

The solutions of Example 1 were stored for a total of 10 weeks at room temperature and at refrigerated conditions. The following results were observed.

Solubility Results after 10 Weeks

| | Room Temperature | Refrigerated |
|---|---|---|
| Solution 1A | Deep blue solution. Lots | Deep blue solution. Lots of |
| | of precipitate on bottom. | precipitate on bottom. |
| Solution 1B | Clear deep blue solution, | Deep blue solution. Some |
| | no precipitate | precipitate on bottom. |
| Solution 1C | Clear deep blue solution, | Clear deep blue solution, |
| | no precipitate | no precipitate |
| Solution 1D | Clear deep blue solution, | Deep blue solution. Some |
| | no precipitate | precipitate on bottom. |

The addition of L-lysine to the solution of peptide copper complex prevented the formation of a precipitate upon storage at these refrigerated conditions. The addition of glycine resulted in a reduced amount of precipitation upon storage at these refrigerated conditions, as did lowering the pH to 4.5.

### Example 4

Solutions of glycyl-L-histidyl-L-lysine:copper(II) complex were prepared at a concentration of 8% with and without the addition of an amino acid or selected preservatives.

| Ingredients | Concentration |
|---|---|
| Solution 4A | |
| Purified Water | q.s. 100% |
| Glycyl-L-Histidyl-L-Lysine: Copper(II) | 8% |
| complex | |
| Imidazolidinyl Urea | 0.3% |
| Methylparaben | 0.1% |
| Propylparaben | 0.03% |
| PH | 6.4 |
| Solution 4B | |
| Purified Water | q.s. 100% |
| Glycyl-L-Histidyl-L-Lysine: Copper(II) | 8% |
| complex | |
| Diazolidinyl Urea | 0.3% |
| Methylparaben | 0.11% |
| Propylparaben | 0.03% |
| Propylene Glycol | 0.56% |
| PH | 6.3 |
| Solution 4C | |
| Purified Water | q.s. 100% |
| Glycyl-L-Histidyl-L-Lysine: Copper(II) | 8% |
| complex | |
| PH | 6.5 |
| Solution 4D | |
| Purified Water | q.s. 100% |
| Glycyl-L-Histidyl-L-Lysine: Copper(II) | 8% |
| complex | |
| PH | 5.0 |
| Solution 4E | |
| Purified Water | q.s. 100% |
| Glycyl-L-Histidyl-L-Lysine: Copper(II) | 8% |
| complex | |
| Glycine | 1.6% |
| Diazolidinyl Urea | 0.3% |
| Methylparaben | 0.11% |
| Propylparaben | 0.03% |
| Propylene Glycol | 0.56% |
| PH | 6.4 |

After formulation the solutions were filtered through 0.2 micron filters and were tested for their ability to inhibit microbial growth. Testing was performed by standard methods as described in the current USP <51>. The microorganisms tested included Canada ablicans (C. albicans), Aspergillus niger (A. niger), Escherichia coli (E. coli), Pseudomonas aeruginosa (P. aeruginosa), and Staphylcoccus aureus (S. aureus). Testing of the samples gave the following results, shown as Examples 4a through 4e. In all cases, the concentrated solutions of peptide copper complex, with or without addition of preservatives, inhibited microbial growth or were toxic to microbial growth.

Example 4a
Inhibition of Microbial Growth by Solution 4A

| | A. | C. | | P. | S. |
|---|---|---|---|---|---|
| | niger | albicans | E. coli | aeruginosa | aureus |
| Day 0 | 170,000 | 490,000 | 600,000 | 440,000 | 500,000 |
| Day 14 | LT 100 | LT 100 | LT 100 | LT 100 | LT 100 |
| Day 28 | LT 100 | LT 100 | LT 100 | LT 100 | LT 100 |

LT = Less Than

Example 4b
Inhibition of Microbial Growth Solution 4B

| | A. | C. | | P. | S. |
|---|---|---|---|---|---|
| | niger | albicans | E. coli | aeruginosa | aureus |
| Day 0 | 170,000 | 490,000 | 600,000 | 440,000 | 500,000 |
| Day 14 | LT 100 | LT 100 | LT 100 | LT 100 | LT 100 |
| Day 28 | LT 100 | LT 100 | LT 100 | LT 100 | LT 100 |

LT = Less Than

Example 4c
Inhibition of Microbial Growth by Solution 4C

| | A. | C. | | P. | S. |
|---|---|---|---|---|---|
| | niger | albicans | E. coli | aeruginosa | aureus |
| Day 0 | 170,000 | 490,000 | 600,000 | 440,000 | 500,000 |
| Day 14 | 57,000 | 58,000 | LT 100 | LT 100 | LT 100 |
| Day 28 | 57,000 | LT 100 | LT 100 | LT 100 | LT 100 |

LT = Less Than

Example 4d
Inhibition of Microbial Growth by Solution 4D

| | A. | C. | | P. | S. |
|---|---|---|---|---|---|
| | niger | albicans | E. coli | aeruginosa | aureus |
| Day 0 | 170,000 | 490,000 | 600,000 | 440,000 | 500,000 |
| Day 14 | LT 100 | 100,000 | LT 100 | LT 100 | LT 100 |
| Day 28 | LT 100 | 200 | LT 100 | LT 100 | LT 100 |

LT = Less Than

Example 4e
Inhibition of Microbial Growth by Solution 4E

| | A. | C. | | P. | S. |
|---|---|---|---|---|---|
| | niger | albicans | E. coli | aeruginosa | aureus |
| Day 0 | 170,000 | 490,000 | 600,000 | 440,000 | 500,000 |
| Day 14 | LT 100 | LT 100 | LT 100 | LT 100 | LT 100 |
| Day 28 | LT 100 | LT 100 | LT 100 | LT 100 | LT 100 |

LT = Less Than

### Example 5

Two solutions of glycyl-L-histidyl-L-lysine:copper(II) complex were prepared having a peptide copper complex concentration of 7.5% and an added L-arginine concentration of 3%. Selected preservatives were also added to one of the solutions.

| Ingredients | Concentration |
|---|---|
| Solution 5A | |
| Purified Water | q.s. 100% |
| Glycyl-L-Histidyl-L-Lysine: Copper(II) | 7.5% |
| complex | |
| L-Arginine | 3.0% |
| PH | 6.3 |
| Solution 5B | |
| Purified Water | q.s. 100% |
| Glycyl-L-Histidyl-L-Lysine: Copper(II) | 7.5% |
| complex | |
| L-Arginine | 3.0% |
| Diazolidinyl Urea | 0.3% |
| Methylparaben | 0.11% |
| Propylparaben | 0.03% |
| Propylene Glycol | 0.56% |
| PH | 6.3 |

After formulation, the solutions were filtered through 0.2 micron filters and stored under three conditions of temperature: ambient (room temperature), refrigerated, and frozen. After 48 hours, the samples were examined (after complete thawing in the case of the frozen samples).

Solubility Results after 48 hours

| | Room | | |
|---|---|---|---|
| Solution | Temperature | Refrigerated | Freeze-Thaw |
| 5A | Clear deep | Clear deep | Clear deep blue |
| | blue solution, | blue solution, | solution with |
| | no precipitate | no precipitate | precipitate on |
| | | | bottom |
| 5B | Clear deep | Clear deep | Clear deep blue |
| | blue solution, | blue solution, | solution with |
| | no precipitate | no precipitate | precipitate on |
| | | | bottom |

The addition of L-arginine to the solution of peptide copper complex prevented the formation of a precipitate upon storage at refrigerated conditions, but not upon freezing followed by thawing.

### Example 6

Solutions 5A and 5B of Example 5 were tested for their ability to inhibit microbial growth. Testing of the sample gave the following results. Both solutions inhibited microbial growth.

Example 6a
Inhibition of Microbial Growth by Solution 5A

| | A. | C. | | P. | S. |
|---|---|---|---|---|---|
| | niger | albicans | E. coli | aeruginosa | aureus |
| Day 0 | 230,000 | 420,000 | 700,000 | 650,000 | 800,000 |
| Day 14 | 53,000 | 140,000 | LT 100 | LT 100 | 1,100 |
| Day 28 | 44,000 | 120,000 | LT 100 | LT 100 | LT 100 |

LT = Less Than

Example 6b
Inhibition of Microbial Growth by Solution 5B

| | A. | C. | | P. | S. |
|---|---|---|---|---|---|
| | niger | albicans | E. coli | aeruginosa | aureus |
| Day 0 | 200,000 | 480,000 | 480,000 | 450,000 | 450,000 |
| Day 14 | 200 | LT 100 | LT 100 | LT 100 | LT 100 |
| Day 28 | 150 | LT 100 | LT 100 | LT 100 | LT 100 |

LT = Less Than

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A composition formed by adding an amino acid to an aqueous solution of a peptide copper complex, with the proviso that the amino acid is not histidine.

2. The composition of claim 1 wherein the molar ratio of the peptide copper complex to the amino acid ranges from about 1:1 to about 3:1.

3. The composition of claim 1 wherein the molar ratio of the peptide copper complex to the amino acid is about 1:1.

4. The composition of claim 1 wherein the concentration of the peptide copper complex ranges from about 1% to about 25% by weight of the solution.

5. The composition of claim 1 wherein the concentration of the peptide copper complex ranges from about 5% to about 15 % by weight of the solution.

6. The composition of claim 1 wherein the concentration of the peptide copper complex ranges from about 7 % to about 10% by weight of the solution.

7. The composition of claim 1 wherein the pH of the solution is adjusted to from about 4.5 to about 7.2.

8. The composition of claim 1 wherein the solution comprises a preservative.

9. The composition of claim 8 wherein the preservative is diazolidinyl urea, benzyl alcohol, phenoxyethanol, imidazolidinyl urea, iodopropynyl butylcarbamate, sodium hydroxymethyglycinate, methylparaben, propylparaben, isopropylparaben, n-butylparaben, isobutylparaben, or a mixture thereof.

10. The composition of claim 9 wherein the solution further comprises propylene glycol.

11. The composition of claim 1 wherein the peptide copper complex is glycyl-L-histidyl-L-lysine:copper(II).

12. The composition of claim 1 wherein the peptide copper complex is L-alanyl-L-histidyl-L-lysine:copper(II).

13. The composition of claim 1 wherein the peptide copper complex is L-valyl-L-histidyl-L-lysine:copper(II).

14. The composition of claim 1 wherein the amino acid is glycine, lysine, arginine, or a mixture thereof.

15. The composition of claim 1 wherein the amino acid is glycine.

16. The composition of claim 1 wherein the amino acid is lysine.

17. The composition of claim 1 wherein the amino acid is arginine.

18. A medical device comprising the composition of claim 1.

19. The composition of claim 1 wherein the composition is in the form of a liquid, lotion, cream, gel, emulsion, or microemulsion.

20. A composition comprising at least one amino acid and an aqueous solution of at least one peptide copper complex, with the proviso that the at least one amino acid is not histidine.

21. The composition of claim 20 wherein the peptide copper complex is glycyl-L-histidyl-L-lysine:copper(II).

22. The composition of claim 20 wherein the peptide copper complex is L-valyl-L-histidyl-L-lysine:copper(II) .

23. The composition of claim 20 wherein the peptide copper complex is L-alanyl-L-histidyl-L-lysine:copper(II).

24. The composition of claim 20 wherein the amino acid is glycine, lysine, arginine, or a mixture thereof.

25. The composition of claim 20 wherein the amino acid is glycine.

26. The composition of claim 20 wherein the amino acid is lysine.

27. The composition of claim 20 wherein the amino acid is arginine.

28. The composition of claim 20, further comprising a ternary complex of the at least one peptide copper complex and the at least one amino acid.

29. A medical device comprising the composition of claim 20.

30. The composition of claim 20 wherein the composition is in the form of a liquid, lotion, cream, gel, emulsion, or microemulsion

31. A method for enhancing the chemical stability of an aqueous solution of a peptide copper complex comprising the step of adding an amino acid to the solution.

32. The method of claim 31 wherein the amino acid is glycine, lysine, arginine, or a mixture thereof, and the molar ratio of the peptide copper complex to the amino acid is at least about 1:1.

33. A method for preventing the precipitation, at ambient temperatures, refrigeration temperatures, or upon thawing after freezing, of a peptide copper complex from an aqueous solution thereof, comprising the step of adding an amino acid to the solution.

34. The method of claim 33 wherein the amino acid is glycine, lysine, argenine, or a mixture thereof, and the molar ratio of the peptide copper complex to the amino acid is at least about 1:1.
